(19) United States
(12) Patent Application Publication
(10) Pub. No.: US 2023/0084129 A1
(43) Pub. Date: Mar. 16, 2023

(54) PHARMACEUTICAL COMPOSITION COMPRISING ESOMEPRAZOLE AND SODIUM BICARBONATE HAVING EXCELLENT RELEASE PROPERTIES

(71) Applicant: Chong Kun Dang Pharmaceutical Corp., Seodaemun-gu, Seoul (KR)

(72) Inventors: Jong Seo Choi, Yongin-si Gyeonggi-do (KR); Min Soo Kim, Yongin-si Gyeonggi-do (KR); Shin Jung Park, Yongin-si Gyeonggi-do (KR)

(73) Assignee: Chong Kun Dang Pharmaceutical Corp., Seodaemun-gu, Seoul (KR)

(21) Appl. No.: 17/801,057

(22) PCT Filed: Feb. 18, 2021
(86) PCT No.: PCT/KR2021/002058

§ 371 Date: Aug. 19, 2022

(30) Foreign Application Priority Data
Feb. 21, 2020 (KR) 10-2020-0021913

(51) Int. Cl. A61K 31/4439; A61K 47/02; A61K 9/08; A61P 1/04

(52) U.S. Cl. CPC A61K 31/4439; A61K 47/02; A61K 9/08; A61P 1/04

(57) ABSTRACT

The present invention relates to a pharmaceutical preparation comprising omeprazole, an enantiomer thereof or a pharmaceutically acceptable salt thereof, and sodium bicarbonate, and a method for preparing the same. Specifically, the present invention relates to a pharmaceutical preparation, wherein a first mixed part comprising esomeprazole comprises 40% by weight or less of sodium bicarbonate based on the total weight of sodium bicarbonate comprised in the preparation, and thus sodium bicarbonate is first dissolved so as to raise pH, and then esomeprazole is dissolved such that the release properties of an active ingredient are improved, and thus the dissolution pattern and bioavailability of a drug can be enhanced.

### TECHNICAL FIELD

The present invention relates to a pharmaceutical preparation comprising omeprazole, an enantiomer thereof or a pharmaceutically acceptable salt thereof, and sodium bicarbonate, and a method for preparing the same. Specifically, the present invention relates to a pharmaceutical preparation comprising a first mixed part comprising esomeprazole and a second mixed part comprising sodium bicarbonate, in which sodium bicarbonate is first dissolved so as to raise pH in the stomach, and then esomeprazole is dissolved such that the release properties of an active ingredient are improved, and thus the dissolution pattern and bioavailability of a drug can be enhanced.

### BACKGROUND ART

Omeprazole has a chemical name of 5-methoxy-2-[(4-methoxy-3,5-dimethyl-2-pyridinyl)methyl]sulfinyl-1H-benzimidazole. Omeprazole exists in the two types of isomers, i.e., R-isomer and S-isomer. S-isomer is known for being remarkably excellent in terms of the therapeutic effect and side effects in comparison with R-isomer. The S-isomer is (S)-5-methoxy-2-[(4-methoxy-3,5-dimethyl-2-pyridinyl)-methyl]sulfinyl-1H-benzimidazole, which is commonly called esomeprazole.

Esomeprazole is a representative proton pump inhibitor (PPI) which is used for the treatment of dyspepsia, peptic ulcer disease, gastroesophageal reflux disease, Zollinger-Ellison syndrome, and the like.

It is well known in the art that omeprazole, especially, esomeprazole, is prone to degradation or transformation in acidic media. More particularly, esomeprazole is known to have a degradation half-life of less than 10 minutes in an aqueous solution having a pH of 3 or less. Therefore, the degradation of esomeprazole is promoted by an acidic compound, and also affected by moisture, heat, organic solvents, and light. Thus, there have been a lot of demands on a stable esomeprazole preparation. In order to solve the stability issue, Korean Patent No. 10-0384960 discloses a method of preparing a pellet comprising a magnesium salt of esomeprazole, followed by enteric coating the same, adding excipients, and formulating as a tablet. The preparation manufactured by the method described above is currently being marketed under the trade name of Nexium.

However, an enteric coated tablet such as Nexium is not suitable for the treatment of diseases requiring immediate therapeutic effect after administration, such as gastric acid-related diseases, because it was designed to be dissolved and absorbed in the intestine while not causing immediate absorption in the stomach.

Korean Patent No. 10-1104349 discloses an enteric coated tablet and capsule in which the problem of the stability and physical properties of omeprazole was improved by preparing a solid dispersion formulation with magnesium oxide and povidone.

Korean Patent Publication No. 10-1996-0003605 discloses a method for preparing a solid dispersion formulation comprising omeprazole as an active ingredient in which beta-cyclodextrin and sodium hydroxide are added as a stabilizing ingredient. However, the invention described in the above patent has a problem of using sodium hydroxide which is harmful to human body. The process of preparing the solid dispersion includes dissolving the active ingredient, omeprazole, in a solvent, and thus during this process, a special stabilizing agent such as sodium hydroxide is required to stabilize omeprazole.

In order to solve these problems, Korean Patent No. 10-0679767 discloses a method of using a buffering agent such as sodium bicarbonate for omeprazole. However, the invention described in the above patent only discloses a combination preparation comprising omeprazole and sodium bicarbonate as a buffering agent at the same time, and does not solve the problem of degradation of omeprazole when omeprazole is disintegrated and dissolved in the gastric juice from these combination preparations.

### PRIOR ART DOCUMENT

### Patent Document

(Patent Document 1) Korean Patent No. 10-0384960
(Patent Document 2) Korean Patent No. 10-1104349
(Patent Document 3) Korean Patent Publication No. 10-1996-0003605
(Patent Document 4) Korean Patent No. 10-0679767

### DISCLOSURE OF THE INVENTION

### Technical Problem

It is an object of the present invention to prevent omeprazole from being degraded in an acidic environment in the stomach to achieve a high dissolution rate and bioavailability.

Therefore, the present invention is to provide a pharmaceutical preparation in which sodium bicarbonate is first dissolved, and then omeprazole is dissolved in order to release omeprazole after neutralizing an acidic environment in the stomach so that omeprazole is not degraded.

In addition, the present invention is to provide a pharmaceutical preparation in which sodium bicarbonate is dissolved, and then omeprazole is immediately dissolved before the neutralized pH in the stomach decreases again so that omeprazole is released in a neutral environment.

### Solution to Problem

The present invention provides a pharmaceutical preparation comprising a first mixed part comprising omeprazole, an enantiomer thereof or a pharmaceutically acceptable salt thereof, and a second mixed part comprising sodium bicarbonate, characterized in that the first mixed part comprises 40% by weight or less of sodium bicarbonate based on the total weight of sodium bicarbonate comprised in the preparation, and when the preparation is dissolved in a solution, sodium bicarbonate is first dissolved, and then the omeprazole, the enantiomer thereof or the pharmaceutically acceptable salt thereof is dissolved.

In one embodiment, the omeprazole, the enantiomer thereof or the pharmaceutically acceptable salt thereof may be esomeprazole, preferably esomeprazole magnesium, and more preferably esomeprazole magnesium salt trihydrate.

In one embodiment, the content of sodium bicarbonate comprised in the first mixed part may be 40% by weight or less based on the total weight of sodium bicarbonate comprised in the preparation, preferably 30% by weight or less, more preferably 20% by weight, and even more preferably 10% by weight or less.

In one embodiment, the first mixed part and the second mixed part may be powders, granules, microgranules, beads, microcapsules, pellets, tablets, or any combination thereof. In addition, the pharmaceutical preparation may be in the form of a single tablet, a multi-layered tablet, or a nucleated tablet, but is not limited thereto.

In one embodiment, the first mixed part and the second mixed part may exist in a state separated from each other, and the presence of the mixed parts in a state separated does not necessarily mean that omeprazole and sodium bicarbonate exist in a state separated from each other.

In one embodiment, the first mixed part may be coated with a coating agent. In this case, the coating agent may be polyvinyl alcohol, povidone, hydroxypropyl cellulose, hydroxypropyl methyl cellulose, polyvinyl acetate, ethyl cellulose, or dimethylaminoethyl methacrylate-methyl methacrylate copolymer, but is not limited thereto.

In addition, the present invention provides the immediate release pharmaceutical preparation, characterized in that when the preparation is dissolved in water, 80% of the omeprazole, the enantiomer thereof or the pharmaceutically acceptable salt thereof is dissolved within 1 hour. Preferably, 90% of the omeprazole, the enantiomer thereof or the pharmaceutically acceptable salt thereof may be dissolved within 1 hour, and 80% may be dissolved within 45 minutes.

The flow through cell dissolution test method of the present invention is defined as a dissolution test method for solid preparations in USP apparatus 4, and is a dissolution test method in which a drug is fixed in a cell and a test solution passes through the cell. It refers to a dissolution test method that can confirm the release rate of a drug while maintaining a sink condition similar to an in vivo condition since a pH of a test solution can be immediately changed.

In addition, the present invention provides the pharmaceutical preparation, characterized in that the pH in the stomach increases within 60 minutes after the single administration of the preparation. Preferably, the pH in the stomach increases within 45 minutes after the single administration of the preparation.

In addition, the present invention provides the pharmaceutical preparation, characterized in that the pH in the stomach increases within 30 minutes after the repeated administration of the preparation. Preferably, the pH in the stomach increases within 25 minutes after the repeated administration of the preparation.

In addition, the present invention provides the pharmaceutical preparation, characterized in that the time (Tmax) to reach the highest concentration of the omeprazole, the enantiomer thereof or the pharmaceutically acceptable salt thereof upon the single administration of the preparation is within 1.5 hours. Preferably, the time to reach the highest concentration of the omeprazole, the enantiomer thereof or the pharmaceutically acceptable salt thereof is within 1 hour, and more preferably within 0.75 hours.

In addition, the present invention provides the pharmaceutical preparation, characterized in that the time to reach the highest concentration of the omeprazole, the enantiomer thereof or the pharmaceutically acceptable salt thereof upon the repeated administration of the preparation is within 1.25 hours. Preferably, the time to reach the highest concentration of the omeprazole, the enantiomer thereof or the pharmaceutically acceptable salt thereof is within 1 hour.

In addition, the present invention provides the pharmaceutical preparation, characterized in that the time to maintain the pH in the stomach of 4 or less upon the administration of the preparation is reduced by 30% or more. In this case, the reduction in the time to maintain the pH in the stomach of 4 or less by 30% or more means that the difference between the fraction of the time to maintain the pH in the stomach of 4 or less during 24 hours before the administration of the preparation and the fraction of the time to maintain the pH in the stomach of 4 or less during 24 hours after the administration of the preparation (the fraction of the time to maintain the pH in the stomach of 4 or less during 24 hours before the administration of the preparation—the fraction of the time to maintain the pH in the stomach of 4 or less during 24 hours after the administration of the preparation) is 30% or more.

In the preparation, upon the single administration of the preparation comprising 20 mg of omeprazole, the reduction rate of the time to maintain the pH in the stomach of 4 or less may be 30% or more, and preferably 35% or more.

In the preparation, upon the single administration of the preparation comprising 40 mg of omeprazole, the reduction rate of the time to maintain the pH in the stomach of 4 or less may be 30% or more, preferably 40% or more, and more preferably 50% or more.

In the preparation, upon the repeated administration of the preparation comprising 20 mg of omeprazole, the reduction rate of the time to maintain the pH in the stomach of 4 or less may be 30% or more, preferably 40% or more, and more preferably 45% or more.

In the preparation, upon the repeated administration of the preparation comprising 40 mg of omeprazole, the reduction rate of the time to maintain the pH in the stomach of 4 or less may be 30% or more, preferably 40% or more, more preferably 50% or more, and even more preferably 60% or more.

The present invention provides a method for preparing a pharmaceutical preparation comprising omeprazole, an enantiomer thereof or a pharmaceutically acceptable salt thereof, and sodium bicarbonate, the method comprising the steps of:

(a) preparing a first mixed part comprising omeprazole, an enantiomer thereof or a pharmaceutically acceptable salt thereof;

(b) preparing a second mixed part comprising sodium bicarbonate;

(c) tableting the first mixed part and the second mixed part by a tablet press machine; and

(d) coating the uncoated tablet with a coating solution and then drying to obtain a coated tablet.

In one embodiment, sodium bicarbonate may be further mixed in the step (a) to prepare a pharmaceutical preparation comprising sodium bicarbonate in a first mixed part. The content of sodium bicarbonate mixed in the step (a) may be 40% by weight or less, preferably 30% by weight or less, more preferably 20% by weight or less, and even more preferably 10% by weight or less based on the total weight of sodium bicarbonate comprised in the preparation.

The present invention provides a method for preparing a pharmaceutical preparation comprising omeprazole, an enantiomer thereof or a pharmaceutically acceptable salt thereof, and sodium bicarbonate, the method comprising the steps of:

(a) coating a core with a first coating solution comprising omeprazole, an enantiomer thereof or a pharmaceutically acceptable salt thereof to prepare a first mixed part;

(b) coating the first mixed part with a second coating solution comprising a coating agent to prepare a coated product;

(c) mixing the coated product with sodium bicarbonate to obtain a mixture;

(d) tableting the mixture to obtain an uncoated tablet; and

(e) coating the uncoated tablet with a third coating solution and then drying to obtain a coated tablet.

In one embodiment, in step (c), sodium bicarbonate may be prepared by a wet granulation and then mixed with the coated product.

In one embodiment, sodium bicarbonate may be further mixed in the step (a) to prepare a pharmaceutical preparation comprising sodium bicarbonate in a first mixed part. The content of sodium bicarbonate mixed in the step (a) may be 40% by weight or less, preferably 30% by weight or less, more preferably 20% by weight or less, and even more preferably 10% by weight or less based on the total weight of sodium bicarbonate comprised in the preparation.

In addition, the present invention provides a method for preparing a pharmaceutical preparation comprising omeprazole, an enantiomer thereof or a pharmaceutically acceptable salt thereof, and sodium bicarbonate, the method comprising the steps of:

(a) kneading omeprazole, an enantiomer thereof or a pharmaceutically acceptable salt thereof with a binder solution to obtain a first mixed part;

(b) mixing the first mixed part with sodium bicarbonate to obtain a final mixture;

(c) tableting the final mixture to obtain an uncoated tablet; and

(d) coating the uncoated tablet with a coating solution and then drying to obtain a coated tablet.

In one embodiment, in the step (a), the omeprazole, the enantiomer thereof or the pharmaceutically acceptable salt thereof may be mixed with sodium bicarbonate and then kneaded with a binder solution to obtain a first mixed part comprising sodium bicarbonate. The content of sodium bicarbonate mixed in the step (a) may be 40% by weight or less, preferably 30% by weight or less, more preferably 20% by weight or less, and even more preferably 10% by weight or less based on the total weight of sodium bicarbonate comprised in the preparation.

In addition, the present invention provides a method for preparing a pharmaceutical preparation comprising omeprazole, an enantiomer thereof or a pharmaceutically acceptable salt thereof, and sodium bicarbonate, the method comprising the steps of:

(a) mixing omeprazole, an enantiomer thereof or a pharmaceutically acceptable salt thereof with sodium bicarbonate and then slugging to obtain a first mixture;

(b) mixing the first mixture with sodium bicarbonate to obtain a final mixture;

(c) tableting the final mixture to obtain an uncoated tablet; and

(d) coating the uncoated tablet with a coating solution and then drying to obtain a coated tablet. The content of sodium bicarbonate mixed in the step (a) may be 40% by weight or less, preferably 30% by weight or less, more preferably 20% by weight or less, and even more preferably 10% by weight or less based on the total weight of sodium bicarbonate comprised in the preparation.

### Effect of the Invention

In the present invention, sodium bicarbonate is first dissolved, and then omeprazole is dissolved, thereby neutralizing an acidic environment in the stomach so that omeprazole is not degraded.

In addition, sodium bicarbonate is dissolved, and then omeprazole is immediately dissolved before the neutralized pH in the stomach decreases again so that omeprazole is not degraded.

This prevents omeprazole from being degraded in an acidic environment in the stomach, so that the pharmaceutical preparation of the present invention can exhibit a high dissolution rate and bioavailability of omeprazole.

In addition, the preparation of the present invention can achieve the above effects regardless of the formulation.

### BRIEF DESCRIPTION OF DRAWINGS

FIG. 1 is a graph showing the average pH value in the stomach after the single administration of the preparation of Example 1 and the reference drug.

FIG. 2 is a graph showing the average pH value in the stomach after the repeated administration of the preparation of Example 1 and the reference drug.

### DETAILED DESCRIPTION FOR CARRYING OUT THE INVENTION

Hereinafter, the embodiments and examples of the present application will be described in detail with reference to the accompanying drawings so that those of ordinary skill in the art to which the present invention belongs may easily practice the present invention. However, the present application can be implemented in various forms and is not limited to the embodiments and examples described herein.

Throughout the specification of the present application, unless otherwise stated, when a certain part “comprises” a certain constituent element, it means that the certain part may further comprise other constituent elements rather than exclude other constituent elements.

As used herein, the term “multi-layered tablet” refers to a preparation consisting of several layers, and includes a laminated structure in which one side of one layer is in contact with one side of another layer and a surrounded structure in which all sides of one layer is in contact with another layer, but is not limited thereto. In the multi-layered tablet, one ingredient is not necessarily included in only one layer, and each layer may be prepared in two, three or more layers in a flat shape or a spherical shape.

As used herein, the term “nucleated tablet” refers to a preparation in the form of surrounding one preparation with another preparation. In this case, one ingredient is not necessarily included in only one layer.

Hereinafter, the present invention is to be described in more detail through the following examples, but the following examples are for illustrative purposes only and are not intended to limit the scope of the present invention.

### Examples 1 to 11: Preparation of Combination Preparation of Esomeprazole and Sodium Bicarbonate

### Example 1

The preparation of Example 1 was prepared according to the following preparation method.

1. Preparation of First Mixed Part (Pellet)

Hydroxypropyl cellulose was added and dissolved in purified water, and then arginine, simethicone, esomeprazole magnesium trihydrate (20 mg to 40 mg as esomeprazole), magnesium oxide, and talc were added and dispersed to prepare a first coating solution. White sugar sphere was added to a fluidized bed coating machine, and the first coating solution was sprayed to prepare a first mixed part.

2. Coating of First Mixed Part

Purified water, polyvinyl alcohol, talc, titanium oxide, glycerol monocaprylocaprate, and sodium lauryl sulfate were added to the preparation tank and dispersed to prepare a second coating solution. The coated product of the first mixed part was added to a fluidized bed coating machine, and the second coating solution was sprayed to coat the first mixed part.

3. Preparation of Second Mixed Part (Simple Mixing)

The coated first mixed part was added to a mixing machine, and sodium bicarbonate (800 mg) was added. In this case, purified water may be included depending on the moisture content. Further, copovidone, crospovidone and sodium stearyl fumarate were added and mixed to form the second mixed part (a part excluding the first mixed part) together with sodium bicarbonate to obtain a final mixture.

4. Tableting

The final mixture was tableted by a tablet press machine (uncoated tablet).

5. Coating

Polyvinyl alcohol, talc, titanium oxide, glycerol monocaprylocaprate, sodium lauryl sulfate, red iron oxide, black iron oxide, yellow iron oxide, and purified water were added to the preparation tank and dissolved to prepare a third coating solution. The uncoated tablets were added to a coating machine, and coated with the third coating solution, and then dried to obtain the coated tablets.

### Example 2

The preparation of Example 2 was prepared in the same manner as in Example 1, except that the following wet granulation process was used instead of simple mixing in the preparation process of the second mixed part of step 3 in the preparation method of Example 1 above.

3. Preparation of Second Mixed Part (Wet Granulation) and Mixing

A binder solution was prepared with copovidone and water in a separate container, and then kneaded with sodium bicarbonate (800 mg), and dried to prepare wet granules of a second mixed part. Thereafter, the first mixed part and the second mixed part were added to a mixing machine, and copovidone, crospovidone, and sodium stearyl fumarate were added and mixed (final mixture).

### Example 3

The preparation of Example 3 (20 mg to 40 mg of esomeprazole, 800 mg of sodium bicarbonate) was prepared according to the following preparation method.

1. Mixing

Esomeprazole magnesium trihydrate and microcrystalline cellulose were added and mixed by a High Speed Mixer.

2. Preparation of First Mixed Part (Wet Granulation)

Hydroxypropyl cellulose was added and dissolved in purified water to prepare a binder solution. The binder solution was added to the mixture, kneaded, and dried to prepare wet granules of the first mixed part.

3. Preparation of Second Mixed Part, Mixing and Lubricating

The wet granules of the first mixed part, sodium bicarbonate, copovidone, and croscarmellose sodium were added to a mixing machine and mixed, and then sodium stearyl fumarate was added and lubricated to prepare a final mixture. In this case, a part excluding the first mixed part forms a second mixed part.

4. Tableting and Coating

The final mixture was tableted by a tablet press machine (uncoated tablet). Polyvinyl alcohol, titanium oxide, polyethylene glycol, talc and purified water were added to the preparation tank and dissolved. The uncoated tablets were added to a coating machine, coated, and then dried to obtain the coated tablets.

### Example 4

The preparation of Example 4 (20 mg to 40 mg of esomeprazole, 800 mg of sodium bicarbonate) was prepared according to the following preparation method.

1. Mixing and Lubricating

Esomeprazole magnesium trihydrate, sodium bicarbonate, magnesium oxide, and crospovidone were added and mixed, and then sodium stearyl fumarate was added and lubricated to obtain a mixture.

2. Preparation of First Mixed Part (Dry Granulation)

The mixture was slugged by a slugging machine to prepare a first mixed part.

3. Preparation of Second Mixed Part, Mixing, and Lubricating

The first mixed part, sodium bicarbonate, copovidone and crospovidone were added and mixed, and then sodium stearyl fumarate was added and lubricated to prepare a final mixture. In this case, a part excluding the first mixed part forms a second mixed part.

4. Tableting and Coating

The final mixture was tableted by a tablet press machine (uncoated tablet). Hydroxypropyl methyl cellulose, titanium oxide, polyethylene glycol and purified water were added to the preparation tank and dissolved. The uncoated tablets were added to a coating machine, coated, and then dried to obtain the coated tablets.

### Example 5

1. Preparation of First Mixed Part

Esomeprazole magnesium trihydrate, mannitol, copovidone, crospovidone, and sodium stearyl fumarate were uniformly mixed to prepare a first mixed part.

2. Preparation of Second Mixed Part

Sodium bicarbonate, copovidone, croscarmellose sodium, magnesium stearate, and sodium stearyl fumarate were uniformly mixed to prepare a second mixed part.

3. Tableting

The first mixed part and the second mixed part were tableted by a tablet press machine (uncoated tablet).

4. Coating

Polyvinyl alcohol, titanium oxide, polyethylene glycol, talc and purified water were added to the preparation tank and dissolved. The uncoated tablets were added to a coating machine, coated, and then dried to obtain the coated tablets.

### Examples 6 and 7

The preparations of Examples 6 and 7 were prepared according to the preparation method of Example 5 above, except that in the preparation process of the first mixed part of step 1 in the preparation method, sodium bicarbonate was further mixed to allow sodium bicarbonate to be included in a first mixed part and a second mixed part. The first mixed part of Examples 6 and 7 comprised 5 and 10% by weight of sodium bicarbonate based on the weight (800 mg) of sodium bicarbonate of the whole preparation, respectively.

### Examples 8 to 12

The preparations of Examples 8 to 12 were prepared according to the preparation method of Example 3 above, except that in the mixing process of step 1 in the preparation method, sodium bicarbonate was further mixed to allow sodium bicarbonate to be included in a first mixed part and a second mixed part. The first mixed part of the preparations of Examples 8 to 12 comprised 10, 30, 40, 50 and 75% by weight of sodium bicarbonate based on the weight (800 mg) of sodium bicarbonate of the whole preparation, respectively.

### Test Example 1

Comparative Dissolution Rate Test According to Content of Sodium Bicarbonate in First Mixed Part

The following test was performed to confirm the change in the dissolution rate and AUC of the preparations according to the formulation, and to confirm the content of sodium bicarbonate in the first mixed part exhibiting excellent dissolution rate and AUC.

The dissolution test was performed on the preparations of Examples 1, 3 and 8 to 12 (20 mg of esomeprazole/800 mg of sodium bicarbonate) prepared above, and the dissolution test and analysis conditions are as follows.

<Dissolution Test Condition>

1) Dissolution method: the Korean Pharmacopoeia, General tests, Dissolution test, Method 3 (Flow Through Cell Method)

2) Dissolution medium: pH 1.2→pH 4.0

3) Dissolution temperature: 37±0.5° C.

4) Flow rate: 2 m/min

5) Test time: pH 1.2 (15 minutes)→pH 4.0 (15 minutes)

6) Cell size: 22.4 mm

<HPLC Analysis Condition>

1) Detector: UV spectrophotometer (measurement wavelength: 302 nm)

2) Column: Capcell Pak C18 (4.6×150 mm, 5 μm) or a column equivalent thereto

3) Injection amount: 20 μL

4) Flow rate: 1.0 m/min

5) Column temperature: constant temperature near 30° C.

6) Sample temperature: constant temperature near 10° C.

7) Mobile phase: a mixture of acetonitrile, a buffer solution at pH 7.3*, and water (350:500:150)

* The buffer solution at pH 7.3 was prepared as follows: 1 mol/L sodium dihydrogen phosphate solution and 0.5 mol/L disodium hydrogen phosphate solution were taken in 10.5 mL and 60 mL, respectively, to be put in an 1 L volumetric flask, followed by filling the flask with purified water up to the calibration mark.

The results of the dissolution test are shown in Table 1.

TABLE 1

| Example | | 1 | 3 | 8 | 9 | 10 | 11 | 12 |
|---|---|---|---|---|---|---|---|---|
| % by weight of | | 0 | 0 | 10 | 30 | 40 | 50 | 75 |
| sodium bicarbonate | | | | | | | | |
| in first mixed part | | | | | | | | |
| % by weight of | | 100 | 100 | 90 | 70 | 60 | 50 | 25 |
| sodium bicarbonate | | | | | | | | |
| in second mixed part | | | | | | | | |
| *Esomeprazole | 5 minutes | 0.52 | 1.00 | 1.00 | 0.96 | 0.96 | 0.92 | 0.91 |
| concentration | 10 minutes | 1 | 0.80 | 0.79 | 0.70 | 0.79 | 0.60 | 0.64 |
| | 15 minutes | 0.75 | 0.58 | 0.58 | 0.54 | 0.57 | 0.30 | 0.27 |
| | 30 minutes | 0.21 | 0.14 | 0.14 | 0.19 | 0.11 | 0.02 | 0.02 |

| | | | | | | | |
|---|---|---|---|---|---|---|---|
| AUC | 15.76 | 15.05 | 15.00 | 14.63 | 14.33 | 9.76 | 9.70 |
| T/R ** | 1 | 0.95 | 0.95 | 0.93 | 0.91 | 0.62 | 0.62 |

*The concentration of esomeprazole is expressed as a ratio of the dissolution rate (ng/mL) at each time point with respect to the dissolution rate (ng/mL) at 10 minutes of the preparation of Example 1.
** R is the AUC of the preparation of Example 1, and T represents the AUC of the preparation of each Example.

As shown in Table 1 above, it was confirmed that the AUC and dissolution rate were reduced as the content of sodium bicarbonate in the first mixed part increased based on the weight (800 mg) of sodium bicarbonate of the whole preparation.

In particular, it was confirmed that in Examples 11 and 12 comprising sodium bicarbonate in the content of 50% and 75% in the first mixed part based on the weight (800 mg) of sodium bicarbonate of the whole preparation, respectively, the AUC and the dissolution rate were rapidly reduced.

Therefore, it can be seen that it is suitable to use 40% by weight or less of sodium bicarbonate in the first mixed part based on the weight (800 mg) of sodium bicarbonate of the whole preparation.

### Test Example 2

Clinical Trial of Combination of Esomeprazole and Sodium Bicarbonate—Measurement of Tmax

In order to compare and evaluate the pharmacokinetic and pharmacodynamic properties and safety after the single administration and the repeated administration of the preparation of Example 1 (20 mg or 40 mg of esomeprazole/800 mg of sodium bicarbonate) and Nexium tablets (D027 20 mg or 40 mg) as the reference drug to healthy adults, the clinical trial was performed in a randomized, open-labeled, repeated administration, 2×2 crossover design as shown in Table 2 below.

TABLE 2

| | Numberof | | | |
|---|---|---|---|---|
| Group | subjects | Period 1 | Period 2 | Washout |
| A | 20 | T | R | at least 7 days or more |
| B | 20 | R | T | at least 7 days or more |

T: 1 tablet of the preparation of Example 1, repeated orally administered once a day for 7 days under fasting conditions
R: 1 tablet of D027, repeated orally administered once a day for 7 days under fasting conditions

The clinical trials for the preparation of Example 1 of 20 mg of esomeprazole and the reference drug of 20 mg were performed separately from the preparation of Example 1 of 40 mg of esomeprazole and the reference drug of 40 mg.

All subjects were supposed to take the investigational drug (R or T) at the same time in the morning, about 1 hour later, start the given standard meal (700-800 kcal, containing 5-25% fat), and end the meal within 20 minutes.

Subjects were subjected to baseline pH monitoring for 24 hours in Period 1, and then administered with the investigational drug once a day for a total of 7 days according to the each assigned group from the first day of Period 1. All subjects were supposed to start the given standard meal about 1 hour after administration of the investigational drug and end the meal within 20 minutes.

After the last dose of Period 1, subjects were subjected to washout for 7 days or more, and then were hospitalized again, and Period 2 of clinical trial was performed. In Period 2 of clinical trial, Subjects were subjected to baseline pH monitoring for 24 hours in the same manner as in Period 1, and then administered with the investigational drug once a day for a total of 7 days according to the assigned group from the first day of Period 2. However, unlike Period 1, the subjects of Group A were administered with the reference drug and the subjects of Group B were administered with the preparation of Example 1 at a certain time, and were supposed to start the given standard meal about 1 hour after administration and end the meal within 20 minutes.

In order to compare the pharmacokinetic properties of the preparation of Example 1 and the reference drug, the pharmacokinetic blood collection after the single administration was performed on the first day of each of Period 1 and Period 2, and the pharmacokinetic blood collection after the repeated administration was performed on the 7th day of each of Period 1 and Period 2, just before administration and 0.17 (=10 min), 0.33 (=20 min), 0.5, 0.75, 1, 1.25, 1.5, 2, 2.5, 3, 3.5, 4, 5, 6, 8, 10, 12 h (18 times for each period) after administration. The concentration of esomeprazole in plasma isolated from the collected blood was measured to analyze the median value (minimum value, maximum value) of the time to reach the highest blood concentration (Tₘₐₓ), and the results are shown in Table 3 below.

TABLE 3
Tmax (h)

| | Single | | Repeated | |
|---|---|---|---|---|
| Dose | Example 1 | D026 | Example 1 | D026 |
| 20/800 mg | 0.50 | 1.50 | 0.75 | 1.25 |
| | (0.33, 1.25) | (1.00, 4.00) | (0.33, 1.50) | (0.75, 5.00) |
| 40/800 mg | 0.50 | 1.50 | 0.75 | 1.25 |
| | (0.33, 1.25) | (1.25, 4.83) | (0.33, 2) | (0.75, 4.83) |

The time to reach the highest blood concentration of the preparation of Example 1 was much shorter than that of the reference drug in case of both of the single administration and the repeated administration of 20 mg and 40 mg doses. Thus, it was confirmed that the preparation of Example 1 can exhibit rapid drug efficacy by rapidly releasing esomeprazole.

### Test Example 3

Results of Clinical Trial of Combination of Esomeprazole and Sodium Bicarbonate—Measurement of pH in the Stomach

In the above clinical trial, the pH in the stomach was measured through pH monitoring for 24 hours. The pH monitoring for 24 hours after the single administration was performed on the first day of each of Period 1 and Period 2, and the pH monitoring for 24 hours after the repeated administration was performed on the 7th day of each of Period 1 and Period 2, and the measurement of the pH in the stomach used MMS Ohmega R pH. The catheter of pH meter was calibrated using a standard solution, and only the catheter and pH test instrument successfully calibrated were prepared to be used for the test of the pH in the stomach for 24 hours. Thereafter, the catheter was sufficiently moistened with lubricating gel or water to reduce foreign body sensation, and then inserted into the stomach through the nasal cavity to measure the pH.

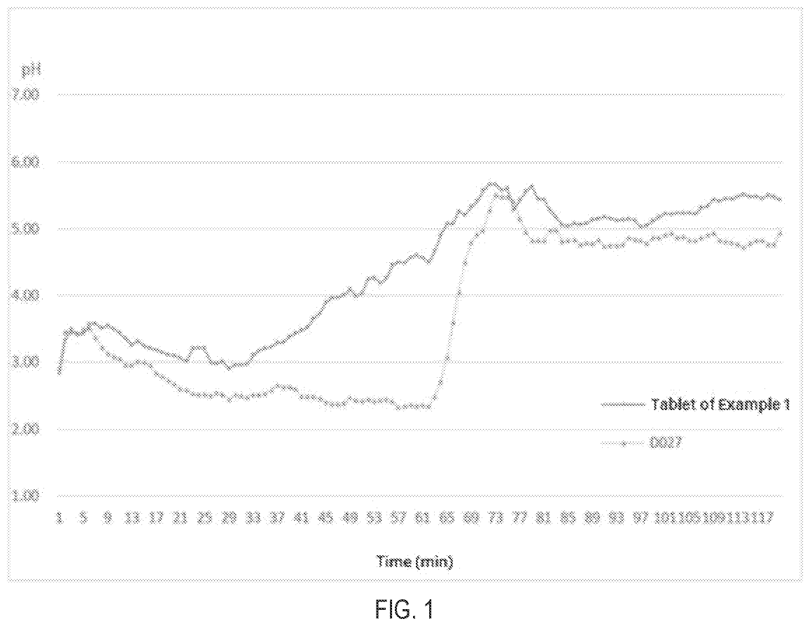

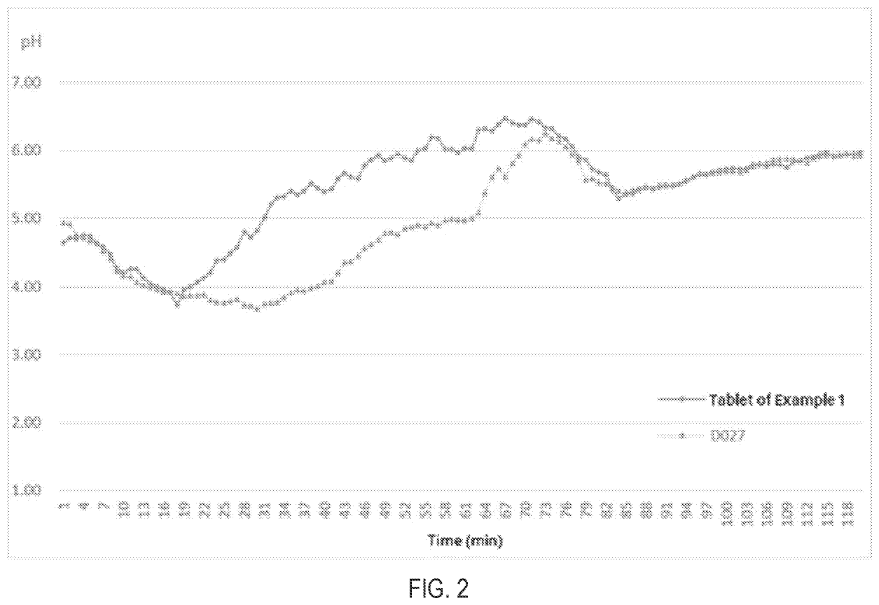

After the single administration and after the repeated administration, the average value of the pH in minutes is shown in FIGS. 1 and 2. As shown in FIG. 1, it was confirmed that for the preparation of Example 1, the pH increased from about 30 minutes after the single dose, whereas for the reference drug, the pH increased from 1 hour after the single dose.

In addition, as shown in FIG. 2, it was confirmed that for the preparation of Example 1, the pH increased from about 20 minutes after the repeated dose, whereas for the reference drug, the pH gradually increased from 30 minutes after the repeated dose.

As a result, it can be seen that the preparation of Example 1 rapidly raises the pH in the stomach when administered.

### Test Example 4

Results of Clinical Trial of Combination of Esomeprazole and Sodium Bicarbonate—Measurement of pH in the Stomach

According to Test Example 3 above, the fraction (%) of the time to maintain pH≤4 of the pH in the stomach observed for 24 hours after the administration of each investigational drug was measured, and the difference compared to the fraction (%) of the time to maintain pH≤4 of the pH in the stomach observed for 24 hours before the administration of the drug was measured. The results are shown in Table 4 below.

TABLE 4

| Dose | Single | Repeated |
|---|---|---|
| 20/800 mg | 36.85% | 49.98% |
| 40/800 mg | 54.36% | 65.81% |

It was confirmed that the time to maintain the pH in the stomach of 4 or less upon the administration of the preparation of Example 1 was reduced.

### Test Example 5

Esomeprazole Dissolution Test

For the tablet of Example 1, the dissolution test was performed in water according to the Korean Pharmacopoeia, General tests, Dissolution test, Paddle method, Method 2 (50 rpm, 900 mL), and the analysis conditions are as follows.

<Analysis condition>

1) Detector: UV spectrophotometer (measurement wavelength: 302 nm)

2) Column: Waters BEH C₁₈ (2.1×50 mm, 1.7 μm)

3) Column temperature: constant temperature near 30° C.

4) Sample temperature: constant temperature near 10° C.

5) Injection amount: 5 μL

6) Flow rate: 0.3 m/min

7) Mobile phase: a mixture of a buffer solution at pH 7.3* and acetonitrile (60:40)

* The buffer solution at pH 7.3 was prepared as follows: 1 mol/L sodium dihydrogen phosphate solution and 0.5 mol/L disodium hydrogen phosphate solution were taken in 10.5 mL and 60 mL, respectively, to be put in an 1 L volumetric flask, followed by filling the flask with purified water up to the calibration mark.

The results of the dissolution test are shown in Table 5.

TABLE 5

| | | Average dissolution rate (%) (mean ± standard deviation) | | | | | |
|---|---|---|---|---|---|---|---|
| Test | Test | 5 | 10 | 15 | 30 | 45 | 60 |
| solution | group | minutes | minutes | minutes | minutes | minutes | minutes |
| Water | Example 1 | 3.1 ± 0.8 | 17.6 ± 3.5 | 38.7 ± 8.4 | 75.2 ± 7.4 | 87.8 ± 4 | 93.3 ± 1.8 |

As described above, it was confirmed that the preparation of Example 1 dissolves 90% or more of esomeprazole within 1 hour and 80% or more of esomeprazole within 45 minutes even in water to show the dissolution pattern of immediate release, and thus can exhibit rapid drug efficacy by rapidly releasing esomeprazole.

1. A pharmaceutical preparation comprising
a first mixed part comprising omeprazole, an enantiomer thereof or a pharmaceutically acceptable salt thereof; and
a second mixed part comprising sodium bicarbonate, characterized in that
the first mixed part comprises 40% by weight or less of sodium bicarbonate based on the total weight of sodium bicarbonate comprised in the preparation, and
when the preparation is dissolved in a solution, sodium bicarbonate is first dissolved, and then the omeprazole, the enantiomer thereof or the pharmaceutically acceptable salt thereof is dissolved.

2. The pharmaceutical preparation according to claim 1, characterized in that the first mixed part comprises 30% by weight or less of sodium bicarbonate based on the total weight of sodium bicarbonate comprised in the preparation.

3. The pharmaceutical preparation according to claim 1, characterized in that the first mixed part and the second mixed part are powders, granules, microgranules, beads, microcapsules, pellets, tablets, or any combination thereof.

4. The immediate release pharmaceutical preparation according to claim 1, characterized in that when the preparation is dissolved in water, 80% of the omeprazole, the enantiomer thereof or the pharmaceutically acceptable salt thereof is dissolved within 1 hour.

5. The immediate release pharmaceutical preparation according to claim 4, characterized in that 90% of the omeprazole, the enantiomer thereof or the pharmaceutically acceptable salt thereof is dissolved within 1 hour.

6. The immediate release pharmaceutical preparation according to claim 4, characterized in that 80% of the omeprazole, the enantiomer thereof or the pharmaceutically acceptable salt thereof is dissolved within 45 minutes.

7. The pharmaceutical preparation according to claim 1, characterized in that the pH in the stomach increases within 60 minutes after the single administration of the preparation.

8. The pharmaceutical preparation according to claim 7, characterized in that the pH in the stomach increases within 45 minutes after the single administration of the preparation.

9. The pharmaceutical preparation according to claim 1, characterized in that the pH in the stomach increases within 30 minutes after the repeated administration of the preparation.

10. The pharmaceutical preparation according to claim 9, characterized in that the pH in the stomach increases within 25 minutes after the repeated administration of the preparation.

11. The pharmaceutical preparation according to claim 1, characterized in that the time to reach the highest concentration of the omeprazole, the enantiomer thereof or the pharmaceutically acceptable salt thereof upon the single administration of the preparation is within 1.5 hours.

12. The pharmaceutical preparation according to claim 11, characterized in that the time to reach the highest concentration of the omeprazole, the enantiomer thereof or the pharmaceutically acceptable salt thereof upon the single administration of the preparation is within 1 hour.

13. The pharmaceutical preparation according to claim 1, characterized in that the time to reach the highest concentration of the omeprazole, the enantiomer thereof or the pharmaceutically acceptable salt thereof upon the repeated administration of the preparation is within 1.25 hours.

14. The pharmaceutical preparation according to claim 13, characterized in that the time to reach the highest concentration of the omeprazole, the enantiomer thereof or the pharmaceutically acceptable salt thereof upon the repeated administration of the preparation is within 1 hour.

15. The pharmaceutical preparation according to claim 1, characterized in that the time to maintain the pH in the stomach of 4 or less upon the administration of the preparation is reduced by 30% or more.

16. The pharmaceutical preparation according to claim 1, characterized in that the omeprazole, the enantiomer thereof or the pharmaceutically acceptable salt thereof is esomeprazole.

17. The pharmaceutical preparation according to claim 1, characterized in that the omeprazole, the enantiomer thereof or the pharmaceutically acceptable salt thereof is esomeprazole magnesium salt.

18. The pharmaceutical preparation according to claim 1, characterized in that the omeprazole, the enantiomer thereof or the pharmaceutically acceptable salt thereof is esomeprazole magnesium trihydrate.
